(19) United States
(12) Patent Application Publication
(10) Pub. No.: US 2021/0213266 A1
(43) Pub. Date: Jul. 15, 2021

(54) Cardiac Cell Microneedle Patch for Treating Heart Diseases

(71) Applicant: NORTH CAROLINA STATE UNIVERSITY, Raleigh, NC (US)

(72) Inventors: Ke CHENG, Raleigh, NC (US); Zhen GU, Los Angeles, CA (US)

(21) Appl. No.: 17/270,897

(22) PCT Filed: Aug. 23, 2019
(86) PCT No.: PCT/US2019/047894

§ 371 Date: Feb. 24, 2021

Related U.S. Application Data
(60) Provisional application No. 62/722,292, filed on Aug. 24, 2018.

(51) Int. Cl. A61M 37/00; A61K 9/00; A61K 35/34; A61P 9/10

(52) U.S. Cl. CPC A61M 37/0015; A61P 9/10; A61K 35/34; A61K 9/0021

(57) ABSTRACT

Disclosed are compositions and methods for delivering cardiac precursor cells to the site of cardiac injury. In one aspect, disclosed herein are microneedle patches for transport of a material across a biological barrier of a subject comprising a plurality of microneedles each having abase end and a tip; a substrate to which the base ends of the microneedles are attached or integrated; and a plurality of cardiac precursor cells (such as, for example, cardiac stem cells), as, well as, methods of using the same.

This Application claims the benefit of U.S. Provisional Application No. 62/722,292, filed on Aug. 24, 2018, which is incorporated herein by reference in its entirety. This invention was made with government support under Grant No. HL123920 awarded by the National Institute of Health. The Government has certain rights in the invention.

### BACKGROUND

Each year, an estimated ˜635,000 Americans have a new coronary attack (defined as first hospitalized myocardial infarction or coronary heart disease death) and ˜300 000 have a recurrent attack, as well as an additional 155,000 silent first myocardial infarctions occur. 36% of MI survivors will develop heart failure (HF), and will be at increased risk for death consequently. To date, no approved therapy has been available to reduce the size of an established scar on the heart. Stem cell therapy aims to alter this fixed trajectory for MI survivors: such as to intervene adverse heart remodeling, to reduce scar size and to actually regenerate viable myocardial tissue. The last one and half decades have witnessed the booming of stem cell therapies for multiple diseases. Deviating from the initial perspective that stem cells exert their therapeutic effects through direct cell differentiation and tissue replacement, the paradigm has shifted as emerging evidence suggesting that most adult stem cell types exert their beneficial effects through paracrine mechanisms, for example, regenerative factors released from stem cells that can promote endogenous repair of the injured myocardium. The notion that injection of heart-derived cardiac stem cells (CSCs) can offer beneficial effect is less assured since mild-moderate MI has been confirmed in recently completed clinical trials. One major hurdle hampering the efficacy of stem cell therapy in the heart is the extremely low cell retention rate after delivery. What is needed are new therapies that do not suffer the drawbacks of CSC injection.

### SUMMARY

Disclosed are methods and compositions related to treating cardiac injury comprising administering a microneedle patch comprising cardiac precursor cells to a subject with the cardiac injury.

In one aspect, disclosed herein are microneedle patches for transport of a material (such as, for example, Adrenomedulin (ADM), Angio-associated migratory protein (AAMP), angiogenin (ANG), angiopoietin-1 (AGPT1), bone morphogenic protein-2 (BMP2), bone morphogenic protein-6 (BMP6), connective tissue growth factor (CTGF), endothelin-1 (EDN1), fibroblast growth factor-2 (FGF2), fibroblast growth factor-7 (FGF7), hepatocyte growth factor (HGF), insulin-like growth factor-1 (IGF-1), interleukin-1 (IL-1), interleukin-6 (IL-6), Kit ligand/Stem cell factor (KITLG (SCF), leukemia inhibitor factor (LIF), macrophage migration inhibitory factor (MIF), matrix metalloproteinase-1 (MMP1), matrix metalloproteinase-2 (MMP2), matrix metalloproteinase-9 (MMP9), macrophage-specific colony-stimulating factor (MCSF), plasminogen activator (PA), platelet-derived growth factor (PDGF), pleiotropin (PTN), secreted frizzled-related protein-1 (SFRP1), secreted frizzled-related protein-2 (SFRP2), stem cell derived factor-1 (SDF-1), thymosin-β4 (TMSB4), tissues inhibitor of metalloproteinase-1 (TIMP1), tissues inhibitor of metalloproteinase-2 (TIMP2), transforming growth factor-β (TGF-β), tumor necrosis factor-α (TNF-α), and/or vascular endothelial growth factor (VEGF)) across a biological barrier of a subject comprising a plurality of microneedles each having a base end and a tip; a substrate to which the base ends of the microneedles are attached or integrated; and a plurality of cardiac precursor cells (such as, for example, cardiac stem cells).

Also disclosed herein are microneedle patches of any preceding aspect, wherein the plurality of microneedles comprises a biocompatible polymer (such as, for example polyvinyl alcohol (PVA)). In one aspect, biocompatible polymer can be crosslinked. Also disclosed herein are microneedle patches of any preceding aspect, wherein the plurality of microneedles have a center-to-center interval of about 200 μm to about 800 μm and/or wherein the plurality of microneedles have a height of about 600 nm to 1.8 μm.

In one aspect, disclosed herein are methods of locally delivering a cardiac precursor cell to a site of cardiac injury comprising providing a microneedle patch for transport of a material across a biological barrier of a subject and administering the microneedle patch to a subject in need thereof to the site of the cardiac injury; wherein the microneedle patch for transport of the material across a biological barrier comprises a plurality of microneedles each having a base end and a tip; a substrate to which the base ends of the microneedles are attached or integrated; and a plurality of cardiac precursor cells attached to the basal surface of the microneedle patch.

Also disclosed herein are method of treating cardiac injury (such as, for example cardiac injury is caused by myocardial infarction, ischemic injury, and ischemic reperfusion injury, pericarditis, acute gastroenteritis, myocarditis, surgery, blunt trauma) comprising administering to a subject with cardiac injury the microneedle patch of any preceding aspect. In one aspect disclosed herein are methods treating a cardiac injury in a subject in need thereof comprising providing a microneedle patch for transport of a material across a biological barrier of a subject comprising: a plurality of microneedles each having a base end and a tip; a substrate to which the base ends of the microneedles are attached or integrated; and a plurality of cardiac precursor cells attached to the basal surface of the microneedle patch; and administering the microneedle patch to a subject in need of treating cardiac injury.

### BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, and 1C show characterization of microneedle (MN) patch integrated with cardiac stem cells (MN-CSCs). FIG. 1A shows a schematic showing the overall design to test the therapeutic benefits of MN-CSCs on infarcted heart. FIG. 1B shows a scanning electron microscope (SEM) image of MN. Scale bar, 500 μm. FIG. 1C shows a representative fluorescent image indicating that DiO-labeled CSCs (green) were encapsulated in fibrin gel and then integrated onto the top surface of MN array (red). Scale bar, 500 μm.

9. FIGS. 2A, 2B, and 2C show characterization of PVA MN. FIG. 2A shows representative fluorescent images of MN. FIG. 2B shows the mechanical strength of MN was determined as 2 N/needle. FIG. 2C shows the integrity of the PVA MN in PBS at day 1, day 3 and day 7. Scale bars, 600 μm.

FIGS. 3A, 3B, 3C, 3D, 3E, 3F, 3G, 3H, 3I, and 3J show the effects of MN-CSCs on neonatal rat cardiomyocytes (NRCMs) function in vitro. FIG. 3A shows a schematic showing the study design to test the effects of MN-CSCs on NRCMs in vitro. FIG. 3B shows Calcein(live)/EthD(dead) staining revealed the viability and morphology of CSCs cultured on the MN patch, and quantitative analysis of CSC viability on day 1, day 3 and day 7. n=3 for each group at each time points. Scale bar, 200 μm. FIG. 3C shows a confocal image indicating that some CSCs (green) escaped from fibrin gel and migrated into the cavity of MN after 3 days in culture. FIG. 3D shows releases of various CSC-secreted factors (namely vascular endothelial growth factor [VEGF], insulin-like growth factor [IGF]-1 and hepatocyte growth factor [HGF]). n=6 for each group at each time point. FIG. 3E shows calcein(live)/EthD(dead) staining revealed the morphology and viability of NRCMs 3 cays in culture with MN-CSC patch. Scale bar, 200 μm. FIG. 3F shows quantitative analysis of NRCM morphology cultured alone or cocultured with MN or MN-CSC patch at Day 3. n=6 for each group. FIG. 3G shows quantitative analysis of cell viability for NRCMs cultured alone or cocultured with MN or MN-CSC patch at day 3. n=3 for each group. FIG. 3H shows time lapse videos revealed co-culture with MN-CSC patch significantly increased cardiomyocyte contractility at day 3. n=6 for each group. FIGS. 31 and 3J show representative fluorescent micrographs and quantitative analysis of NRCMs stained with alpha sarcomeric actin (green) and proliferation marker Ki67 (red), cultured alone or with MN or MN-CSC patch. n=3 for each group. Scale bar, 50 μm. All data are mean ±s.d. Comparisons between any two groups were performed using two-tailed unpaired Student's t-test. Comparisons among more than two groups were performed using one-way ANOVA followed by post hoc Bonferroni test. * indicated P<0.05.

FIGS. 4A, 4B, 4C, 4D, 4E, 4F, 4G, and 4H show that the MN-CSC patch reduces apoptosis and promotes angiomyogenesis in the post-MI heart. FIG. 4A shows a schematic showing the overall design of animal study to test the therapeutic benefits of MN-CSCs in a rat model of myocardial infarction. FIG. 4B shows placement of a MN-CSC patch on the rat heart. Red circle line indicated the area of the MN-CSC patch. FIG. 4C shows H&E staining indicating the presence of MN-CSC patch on the infarcted heart. Scale bar, 1mm FIG. 4D shows fluorescent image showing Cy5.5-labeled MNs (red) can be readily detected on the heart (green) 7 days after the transplantation. Scale bar, 400 μm. FIG. 4E shows representative fluorescent micrographs showing the presence of CD68P^{pos} cells (green) in the Control MI heart or hearts treated with MN or MN-CSC patch at day 7. The numbers of CD68P^{pos} cells were quantified. n=3 hearts for each group. Scale bar, 200 μm. FIG. 4F shows representative fluorescent micrographs showing the presence of TUNELP^{pos} apoptotic cells (green) in the MI hearts treated alone or treated with MN or MN-CSC patch at day 7. The numbers of TUNELP^{pos} apoptotic cells were quantified. n=3 for each group. Scale bar, 100 μm. FIG. 4G shows representative fluorescent micrograph showing the presence of Ki67-positive cardiomyocyte nuclei (red) in the MI hearts treated with MN-CSC on day 7. The numbers of Ki67-positive nuclei were quantified in MI−, MI+MN− or MI+ MN-CSC treated hearts. n=3 for each group. Scale bar, 200 μm. FIG. 4H shows representative fluorescent micrograph showing the presence of alpha smooth muscle actin (α-SMA, green) in the MI hearts treated with MN-CSC on day 7. The numbers of α-SMA positive vasculatures were quantified in MI−, MI+ MN− or MI+ MN-CSC treated hearts. n=3 for each group. Scale bar, 200 μm. All data are means ±s.d. Comparisons among more than two groups were performed using one-way ANOVA followed by post hoc Bonferroni test. * indicated P<0.05.

FIGS. 5A, 5B, 5C, and 5D show Local T cell immune response in immunocompetent rat treated with a MN-CSC patch. FIG. 5A shows representative fluorescent images showing the presence of infiltrated CD8ₚₒₛ T cells (green) in MN-CSC patched heart at Day 7. Scale bar, 200 μm. FIG. 5B shows representative fluorescent images showing the presence of infiltrated CD3ₚₒₛ T cells (green) in MN-CSC patched heart at Day 7. Scale bar, 200 μm. FIG. 5C shows quantitative analysis of CD8ₚₒₛ T cells in MN-CSC patched heart or normal heart at day 7. n=3 animals per group. FIG. 5D shows quantitative analysis of CD3ₚₒₛ T cells in MN-CSC patched heart or normal heart at day 7. All data are mean ±s.d. Comparisons between any two groups were performed using two-tailed unpaired Student's t-test.

FIGS. 6A, 6B, 6C, 6D, 6E, and 6F show that MN-CSC ameliorated ventricular dysfunction and promoted cardiac repair in a rat model of heart attack. FIG. 6A shows representative Masson's trichrome-stained myocardial sections 3 weeks afterward in MI, MI+MN, MI+CSC and MI+MN-CSC groups. In this staining blue=scar tissue and red=viable myocardium. Snapshots=high magnification images of the black box area. FIG. 6B shows a snapshot of M mode detection exhibited the wall motion of different treatments. FIG. 6C and 6D show quantitative analyses of infarct wall thickness (6C) and viable tissue in risk area (6D) from the Masson's trichrome images. n=6 animals per group. FIGS. 6E and 6F show LVEF was measured by echocardiography at baseline (4 h after MI) and 3 weeks afterward in MI, MI+MN, MI+CSC and MI+MN-CSC groups. n=6 animals per group. All data are means ±s.d. Comparisons among more than two groups were performed using one-way ANOVA followed by post hoc Bonferroni test. * indicated P<0.05 when compared with MI group; # indicated P<0.05 when compared with MI+MN group; & indicated P<0.05 when compared with MI+CSC group.

FIGS. 7A, 7B, 7C, and 7D show MN-CSC therapy protects cardiac morphology and reduces fibrosis in a rat model of MI. FIG. 7a shows representative Masson's trichrome-stained myocardial sections 3 weeks after treatment (blue=scar tissue and red=viable myocardium). FIGS. 7B, 7C, and 7D show quantitative analyses of infarct size (7b), viable tissue in risk area (7c), and infarct wall thickness (7d) from the Masson's trichrome-stained images. n=5 animals per group. All data are means ±s.d. Comparisons between two groups were performed with two-tailed Student's t-test. * indicated P<0.05. **indicated P<0.005.

15. FIGS. 8A, 8B, 8C, and 8D show that the swine model of MI was successfully created through LAD ligation. FIG. 8A shows representative pictures of MI model creation via LAD ligation (left) and MN-CSC cardiac patch transplantation via suture (right). FIG. 8B shows the serum concentration of cardiac troponin I (cTnl) in animals of the MI only group and MN-CSC patch transplanted group were measured through blood draw before MI, 24 h and 48 h after MI. All data are means ±s.d. n=3 animals per group. *indicated P<0.05 when compared with baseline (before MI) and 48 h after MI. Red bar=MI only group; blue bar=MN-CSC patch transplanted group. FIG. 8C shows quantitative analyses of infarct size at 48 h after MI through calculation. All data are means ±s.d. n=3 animals per group. NS indicated P>0.05 when compared between two groups. gray bar=MI only group; black bar=MN-CSC patch transplanted group. FIG. 8D shows macroscopic TTC staining images revealing infarct area on multiple slices of an infarcted pig heart.

FIGS. 9A, 9B, 9C, 9D, 9E, 9F, 9G, 9H, 9I, and 9J show that MN-CSC ameliorated ventricular dysfunction and promoted cardiac repair in swine model of MI. FIGS. 9A, 9B, and 9C show LVEFs determined by echocardiography at baseline (9a) (4 h post infarct) and endpoint (9b) (48 h post-infarct). The treatment effects calculated as the change of LVEFs from end point to baseline (9c). FIGS. 9D, 9E, and 9F shows FSs also determined by echocardiography at baseline (9d) (4 h post infarct) and endpoint (9e) (48 h post-infarct). The treatment effects calculated as the change of FSs from end point to baseline (9f). All data are means ±s.d. n=3 animals per group. * indicated P<0.05 when compared between two groups. gray bar=MI only group; black bar=MN-CSC patch transplanted group. FIGS. 9G, 9H, 9I, and 9J show ALT (9g), AST (6h), Creatinine (9i) and BUN (9j) were evaluated and compared between baseline (before MI) and endpoint (48 h post MI). All data are means ±s.d. n=3 animals per group. NS indicated P>0.05. Red=MI only group; blue=MN-CSC patch transplanted group.

### DETAILED DESCRIPTION

Before the present compounds, compositions, articles, patches, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The terms “about” and “approximately” are defined as being “close to” as understood by one of ordinary skill in the art. In one non-limiting embodiment, the terms are defined to be within 10% of the associated value provided. In another non-limiting embodiment, the terms are defined to be within 5%. In still another non-limiting embodiment, the terms are defined to be within 1%.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., Type 1 diabetes). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years. assured

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular microneedle patch is disclosed and discussed and a number of modifications that can be made to a number of molecules including the microneedle patch are discussed, specifically contemplated is each and every combination and permutation of microneedle patch and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Within 24 hours of delivery, over 90% of injected stem cells are typically lost, regardless of the delivery route (such as intracoronary or intramyocardial injection). Bioengineering approaches, including injectable biomaterials, cardiac patches and magnetic/molecular targeting can be used to improve cell engraftment rate. The cardiac patch strategy can drastically improve cell retention. However, the challenge remains to integrate the transplanted biomaterials/stem cells construct with the host myocardium.

Described herein is an innovative microneedle patch integrated with cardiac stem cells (MN-CSCs) for therapeutic heart regeneration. The painless MN patches have been established as an effective transcutaneous delivery device for transporting a variety of therapeutics. In this strategy, the MN-CSC system offers unique advantages over conventional cardiac patches: the MNs serve as the channels to allow the communication between the patch and the host myocardium as the transplanted patch can get nutrients from the heart while releasing the stem cell factors to repair the heart. In a rat model of myocardial infarction, it was demonstrated the MN-CSC patch can promote healing after acute MI by promoting angiomyogenesis, reduction of scar size, and augment of cardiac functions. Accordingly, disclosed herein are microneedle patches for the transport of a cardiac precursor cells across a biological barrier of a subject.

Thus, in one aspect, disclosed herein are devices (for example, microneedle patches) for transport of cardiac precursor cells across a biological barrier of a subject comprising a plurality of microneedles each having a base end and a tip; a substrate to which the base ends of the microneedles are attached or integrated; and a plurality of particles comprising cardiac precursor cells. As used herein, cardiac progenitor cells (CPC) refers to any non-terminally differentiated cell including primitive and early committed cardiac cells that can give rise to further differentiated cardiac lineage cells including, but not limited to totipotent stem cells, pluripotent stem cells, multipotent stem cells, mesenchymal stem cells, adult stem cells, and/or cardiac stem cells (CSC). The CPCs can be obtained from a donor source such as an autologous donor (i.e., the recipient), syngeneic donor source, histocompatible allogenic source, histocompatible xenogenic source, or cell line. It is understood and herein contemplated that one manner in which the therapeutic effects of the CPC is observed is through secretion of a material (such as, for example, factors including, but not limited to Adrenomedulin (ADM), Angio-associated migratory protein (AAMP), angiogenin (ANG), angiopoietin-1 (AGPT1), bone morphogenic protein-2 (BMP2), bone morphogenic protein-6 (BMP6), connective tissue growth factor (CTGF), endothelin-1 (EDN1), fibroblast growth factor-2 (FGF2), fibroblast growth factor-7 (FGF7), hepatocyte growth factor (HGF), insulin-like growth factor-1 (IGF-1), interleukin-1 (IL-1), interleukin-6 (IL-6), Kit ligand/Stem cell factor (KITLG (SCF), leukemia inhibitor factor (LIF), macrophage migration inhibitory factor (MIF), matrix metalloproteinase-1 (MMP1), matrix metalloproteinase-2 (MMP2), matrix metalloproteinase-9 (MMP9), macrophage-specific colony-stimulating factor (MCSF), plasminogen activator (PA), platelet-derived growth factor (PDGF), pleiotropin (PTN), secreted frizzled-related protein-1 (SFRP1), secreted frizzled-related protein-2 (SFRP2), stem cell derived factor-1 (SDF-1), thymosin-β4 (TMSB4), tissues inhibitor of metalloproteinase-1 (TIMP1), tissues inhibitor of metalloproteinase-2 (TIMP2), transforming growth factor-β (TGF-β), tumor necrosis factor-α (TNF-α), and/or vascular endothelial growth factor (VEGF)) to the injured tissue. The microneedle patch allows these secreted factors to pass through the needle directly into the tissue. In one aspect, the CPC can be placed on the basal side of the microneedle patch. As one purpose of the CPC being used in the disclosed microneedle patches the secretion of factors to the injured tissue, it is understood and herein contemplated that any non-stem cell or exosome capable of secreting one of the disclosed factors to the injured tissue can be used in combination with or alternatively to the CPC in the disclosed microneedle patches. Thus, disclosed herein are microneedle patches and methods of their use, wherein the microneedle patch comprises stem cell exosomes and/or non-stem cells. In one aspect, the disclosed microneedles can also be fabricated already comprising stem cell factors, or drugs beneficial to tissue healing in addition to or alternatively to the presence of CPCs, stem cell exosomes, or non-stem cells.

To not only protect the cargo of the cardiac precursor cells but also allow for the attachment of the cardiac precursor cell cargo to the microneedles, it is contemplated herein that the cardiac precursor cells can be encapsulated by a substrate and integrated onto the surface of the microneedle. For example, the substrate can be a Fibrin gel, poly(vinyl alcohol) (PVA) gel, and/or PVA methacrylate (m-PVA) gel that can be crosslinked to the core of the microneedle.

It is understood and herein contemplated plurality of microneedles can comprise a biocompatible polymer (such as, for example, methacrylated hyaluronic acid (m-HA) or PVA). In one aspect, biocompatible polymer can be crosslinked. Such polymers can also serve to slowly release the CPC into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the particle contains biocompatible polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide₅ collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

In one aspect, the disclosed patches can comprise a plurality of microneedles, wherein the plurality of microneedles have a center-to-center interval of about 200 um to about 800 um, for example a center to center interval of about 200, 225, 250, 275, 300, 325, 350, 375, 400, 425, 450, 475, 500, 525, 550, 575, 600, 625, 650, 675, 700, 725, 750, 775, or 800 μm.

The disclosed microneedles can have a cylindrical or conical shape having a base comprising a diameter that is the same or broader than the diameter at the needle tip. In one aspect the diameter of the microneedle at the base can be 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, or 800 μm. In one aspect the tip of the needle can have a diameter of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 μm.

It is also understood and herein contemplated that the disclosed plurality of microneedles in the disclosed patches is effective when the length of the needle is sufficiently long to reach desired tissues below the dermal layer. Thus, in one aspect, disclosed herein are patches wherein the plurality of microneedles has a height of about 600 nm to 1.8 μm. For example, the plurality of microneedles can have a height of about 600, 650, 700, 750, 800, 850, 900, 950 nm, 1.0, 1.1, 1.2, 1.3, 1.4, 1.5, 1.6, 1.7, or 1.8 μm.

In one aspect, the microneedles on the disclosed patch can be randomly arranged or arranged in an array such as a 4×5, 5×5, 5×6, 6×6, 6×8, 7×7, 8×8, 9×9, 10×10, 11×11, 12×12, 13×13, 14×14, 15×15, 16×16, 17×17, 18×18, 19×19, 20×20, 21×21, 22×22, 23×23, 24×24, 25×25, 30×30, 40×40, or 50×50 microneedle array.

The patches can be any size and shape (circle, oval, rectangle, square, trapezoid, rhombus, or triangle) appropriate for the application and special requirements of the tissue or site receiving the patch including but not limited to a circle with a 1, 2, 3, 4, 5, 6, 7, 8, 9, 10, 11, 12, 13, 14, 15, 16, 17, 18, 19, 20, 21, 22, 23, 24, or 25 mm diameter ora square or rectangle with a 2×2, 3×3, 4×4, 4×5, 5×5, 5×6, 6×6, 6×8, 7×7, 8×8, 9×9, 10×10, 11×11, 12×12, 13×13, 14×14, 15×15, 16×16, 17×17, 18×18, 19×19, 20×20, 21×21, 22×22, 23×23, 24×24, 25×25, 30×30, 40×40, or 50×50 mm² shape.

In one aspect, disclosed herein are methods of locally delivering a cardiac precursor cell to a site of cardiac injury comprising providing a microneedle patch for transport of a material (such as, for example, factors including, but not limited to Adrenomedulin (ADM), Angio-associated migratory protein (AAMP), angiogenin (ANG), angiopoietin-1 (AGPT1), bone morphogenic protein-2 (BMP2), bone morphogenic protein-6 (BMP6), connective tissue growth factor (CTGF), endothelin-1 (EDN1), fibroblast growth factor-2 (FGF2), fibroblast growth factor-7 (FGF7), hepatocyte growth factor (HGF), insulin-like growth factor-1 (IGF-1), interleukin-1 (IL-1), interleukin-6 (IL-6), Kit ligand/Stem cell factor (KITLG (SCF), leukemia inhibitor factor (LIF), macrophage migration inhibitory factor (MIF), matrix metalloproteinase-1 (MMP1), matrix metalloproteinase-2 (MMP2), matrix metalloproteinase-9 (MMP9), macrophage-specific colony-stimulating factor (MCSF), plasminogen activator (PA), platelet-derived growth factor (PDGF), pleiotropin (PTN), secreted frizzled-related protein-1 (SFRP1), secreted frizzled-related protein-2 (SFRP2), stem cell derived factor-1 (SDF-1), thymosin-β4 (TMSB4), tissues inhibitor of metalloproteinase-1 (TIMP1), tissues inhibitor of metalloproteinase-2 (TIMP2), transforming growth factor-β (TGF-β), tumor necrosis factor-α (TNF-α), and/or vascular endothelial growth factor (VEGF)) as disclosed herein across a biological barrier of a subject and administering the microneedle patch to a subject in need thereof to the site of the cardiac injury. For example disclosed herein are methods of locally delivering a cardiac precursor cell to a site of cardiac injury comprising providing a microneedle patch for transport of a material across a biological barrier of a subject and administering the microneedle patch to a subject in need thereof to the site of the cardiac injury; wherein the microneedle patch for transport of the material across a biological barrier comprises a plurality of microneedles each having a base end and a tip; a substrate to which the base ends of the microneedles are attached or integrated; and a plurality of cardiac precursor cells attached to the basal surface of the microneedle patch.

It is understood and herein contemplated that the disclosed microneedle patches can provide therapeutic benefit to the site of cardiac injury. Accordingly, disclosed herein are method of treating cardiac injury comprising administering to a subject with cardiac injury any of the microneedle patches disclosed herein. In one aspect disclosed herein are methods treating a cardiac injury in a subject in need thereof comprising providing a microneedle patch for transport of a material across a biological barrier of a subject comprising: a plurality of microneedles each having a base end and a tip; a substrate to which the base ends of the microneedles are attached or integrated; and a plurality of cardiac precursor cells attached to the basal surface of the microneedle patch; and administering the microneedle patch to a subject in need of treating cardiac injury.

It is understood and herein contemplated that cardiac injury can be caused by many different means including microbial disease, inflammatory disease, medical procedures, blunt trauma, and/or other cardiac maladies, for example, cardiac injury can be infarct injury from myocardial infarction, ischemic injury, and ischemic reperfusion injury, pericarditis, acute gastroenteritis, myocarditis, surgery, blunt trauma, heart failure, congenital heart defects, cardiac tumor, and cardiac arrhythmia. Accordingly, in one aspect, disclosed herein are method of treating cardiac injury (such as, for example cardiac injury is caused by myocardial infarction, ischemic injury, and ischemic reperfusion injury, pericarditis, acute gastroenteritis, myocarditis, surgery, blunt trauma) in a subject comprising administering to the subject the microneedle patch disclosed herein. Thus, for example, disclosed herein are methods of treating injury from myocardial infarction in a subject comprising administering to the subject a microneedle patch comprising cardiac precursor cells. In one aspect disclosed herein are methods treating a cardiac injury in a subject in need thereof comprising providing a microneedle patch for transport of a material across a biological barrier of a subject comprising: a plurality of microneedles each having a base end and a tip; a substrate to which the base ends of the microneedles are attached or integrated; and a plurality of cardiac precursor cells attached to the basal surface of the microneedle patch; and administering the microneedle patch to a subject in need of treating cardiac injury.

“Treat,” “treating,” “treatment,” and grammatical variations thereof as used

herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

It is understood and herein contemplated that a cardiac injury (such as, for example cardiac injury is caused by myocardial infarction, ischemic injury, and ischemic reperfusion injury, pericarditis, acute gastroenteritis, myocarditis, surgery, blunt trauma) is optimally treated as quickly as possible to minimize the damage to cardiac tissue caused by the injury. However, the disclosed CPC comprising microneedles can be used to treat cardiac injuries that occurred, hours, days, weeks, or months prior to the application of the microneedle patch. Accordingly, in one aspect, disclosed herein are methods of treating cardiac injury in a subject comprising administering to the subject a microneedle patch comprising cardiac precursor cells, wherein the microneedle patch is contacted to the site of the cardiac injury at the time of injury, 1, 2, 3, 4, 5, 6, 7, 8,9 10, 11, 12, 13, 14, 15, 16, 17, 18, 19, 20, 21, 22, 23, 24, 31, 32, 33, 34, 35, 36, 37, 38, 39, 40, 41, 42, 43, 44, 45, 46, 47, 48, 60 hours, 3, 4, 5, 6, 7, 8, 9, 10, 11, 12, 13, 14 days, 3, 4, 5, 6, 7, 8 weeks, 2, 3, 4, 5, 6, 7, 8, 9, 10, 11, 12 months after the cardiac injury. In some aspects where the injury is expected as the result of a medical procedure, the microneedle patch can be applied prophylactically 1, 2, 3, 4, 5, 6, 7, 8,9 10, 11, 12, 13, 14, 15, 16, 17, 18, 19, 20, 21, 22, 23, 24, 31, 32, 33, 34, 35, 36, 37, 38, 39, 40, 41, 42, 43, 44, 45, 46, 47, 48, 60 hours prior to the injury.

It is understood and herein contemplated that the disclosed methods of direct application of a microneedle patch to a site of injury to an internal organ can be effective in areas of treatment beyond cardiac injury. For example, the disclosed methods can be adapted to the application of pancreatic islet cells or islet cell precursors for the treatment of diabetes (for example via direct attachment of islet cells to the liver or the pancreas), the administration of chimeric antigen receptor (CAR) T cells, tumor infiltrating lymphocytes (TILs), and/or marrow-infiltrating lymphocytes (MILs) for the treatment of cancer directly at the site of a malignant growth, hepatocytes administered at the site of hepatic injury, as well as the administration of osteoclast, osteoblasts, or precursors of said cells for the treatment of bone injuries. Additionally, totipotent stem cells, pluripotent stem cells, multipotent stem cells, mesenchymal stem cells, adult stem cells, and/or stem cell exosomes can be used in the patches in these methods. In each case the administration of the microneedle patch offers significant advantages over adoptive transfer of cells avoiding the initial loss of transferred cells and ultimate low retention rate of the transferred cells. As with the cardiac applications disclosed above, the disclosed methods of treatment of cancer, diabetes, hepatic injury, and bone injury utilize precursor cells to deliver material (such as, for example, factors including, but not limited to Adrenomedulin (ADM), Angio-associated migratory protein (AAMP), angiogenin (ANG), angiopoietin-1 (AGPT1), bone morphogenic protein-2 (BMP2), bone morphogenic protein-6 (BMP6), connective tissue growth factor (CTGF), endothelin-1 (EDN1), fibroblast growth factor-2 (FGF2), fibroblast growth factor-7 (FGF7), hepatocyte growth factor (HGF), insulin-like growth factor-1 (IGF-1), interleukin-1 (IL-1), interleukin-6 (IL-6), Kit ligand/Stem cell factor (KITLG (SCF), leukemia inhibitor factor (LIF), macrophage migration inhibitory factor (MIF), matrix metalloproteinase-1 (MMP1), matrix metalloproteinase-2 (MMP2), matrix metalloproteinase-9 (MMP9), macrophage-specific colony-stimulating factor (MCSF), plasminogen activator (PA), platelet-derived growth factor (PDGF), pleiotropin (PTN), secreted frizzled-related protein-1 (SFRP1), secreted frizzled-related protein-2 (SFRP2), stem cell derived factor-1 (SDF-1), thymosin-β4 (TMSB4), tissues inhibitor of metalloproteinase-1 (TIMP1), tissues inhibitor of metalloproteinase-2 (TIMP2), transforming growth factor-13 (TGF-β), tumor necrosis factor-α (TNF-α), and/or vascular endothelial growth factor (VEGF)). Thus, in one aspect, the disclosed microneedle patches for use in the disclosed methods, can include in the addition to or in the alternative to any of the cells disclosed herein, a coating or other delivery mechanism for the disclosed material or a therapeutic drug that can promote tissue healing.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, patches and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Results

(1) Characterization of Microneedle Array.

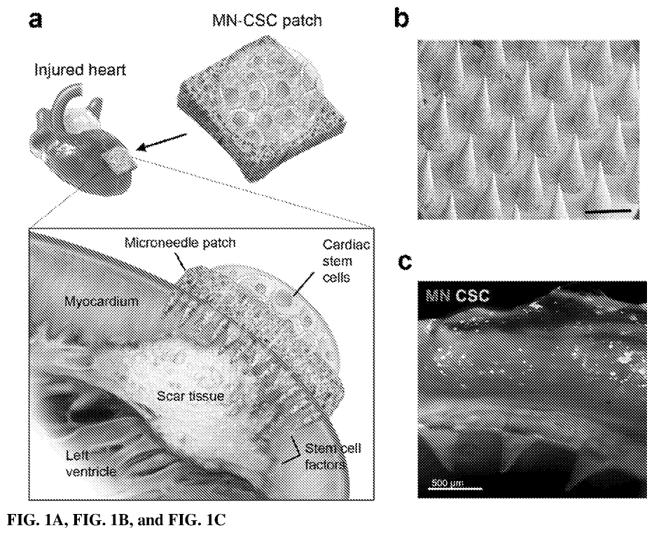

The biochemical design and work model of MN-CSCs were outlined in FIG. 1a. Briefly, MN was fabricated from an aqueous solution of biocompatible polymer poly (vinyl alcohol) (PVA) via a micromolding approach. The PVA is 99% hydrolyzed and has a molecular weight of 89, 000-98, 000 g/mol. The prepared MN array patch was a 12×12 mm² patch with a 20×20 MN array. The needle had a conical shape with a diameter of 300 um at the base, 5 um at the tip, and a height of 600 um as confirmed with scanning electron microscope (SEM) and fluorescence microscopy (FIGS. 1b and 2a). The mechanical strength of MN was determined as 2 N/needle (FIG. 2b), which allowed for sufficient skin insertion without breaking. Meanwhile, the integrity of the PVA MN maintained without obvious deformation in PBS during day 1, day 3 and day 7 (FIG. 2c).

(2) Biocompatibility of the Microneedle Array with CSCs and Cardiomyocytes.

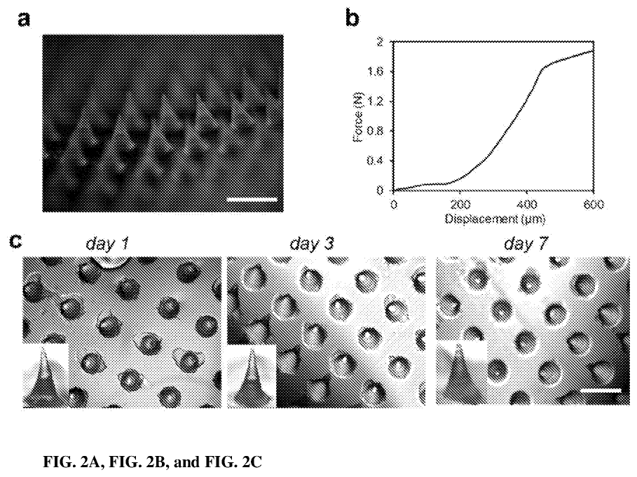

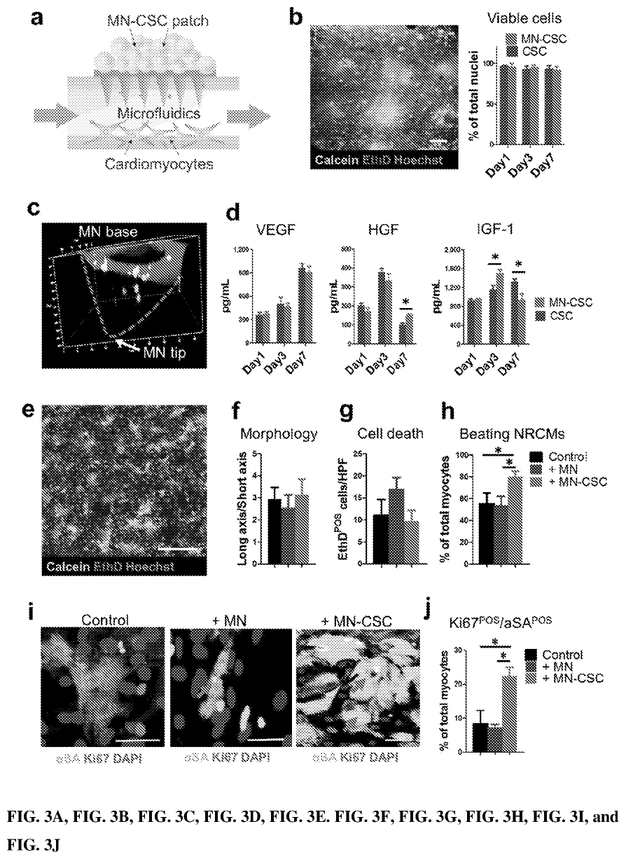

1×10⁵ rat CSCs were encapsulated in the fibrin gel and then integrated onto the surface of MN array (FIG. 1c). The porous structure of MN allowed the release of CSC-factors through the polymeric needles. The integrated MN-CSC patch was cultured by positioning into a microfluidic channel with IMDM media (FIG. 3a). Live/dead staining revealed excellent viability of CSCs on day 3 in culture, and quantitative analysis indicated that the viability of CSCs in fibrin gel were not compromised when co-cultured on MN array on day 1, day 3 and day 7 (FIG. 3b). Confocal imaging results revealed the distribution of CSCs (green) in the MN (red) patch after 3 days in culture (FIG. 3c). Z-stack confocal microscopy reconstruction indicated that some CSCs (green) had escaped from the fibrin gel and migrated into the MN cavity (FIG. 3c). Since the majority of beneficial effects of CSC therapy was through their secretion, ELISA was used to measure the concentrations of various CSC-secreted factors, namely, vascular endothelial growth factor lVEGF1, hepatocyte growth factor [HGF], and Insulin-like Growth Factor [IGF-1] in the underneath media. The factors can be detected, and their release profiles were similar to that of CSCs cultured on the tissue culture plate (TCP) at various time points (FIG. 3d). The biocompatibility of MN-CSC patch was tested in vitro with isolated neonatal rat cardiomyocytes (NRCMs). NRCMs were cultured with the presence of a MN or MN-CSC patch in the media. A solitary NRCM culture was included as a negative control. Live/dead assay indicated the viability of NRCMs (FIG. 3e), and quantitative analysis indicated that the morphology and viability of NRCMs were not compromised when co-cultured with a MN or MN-CSC patch (FIGS. 3f and 3g). Moreover, time lapse videos revealed that coculture with a MN-CSC patch significantly increased cardiomyocyte contractility (FIG. 3h). In addition, MN-CSCs robustly promoted proliferation (as indicated by Ki67-positive nuclei) of cardiomyocytes (FIGS. 3i and 3j). Collectively, these data suggested that the MN patch was nontoxic to cardiomyocytes and the MN-CSC patch promoted cardiomyocyte functions.

(3) MN-CSC Therapy in a Rat Model of Myocardial Infarction

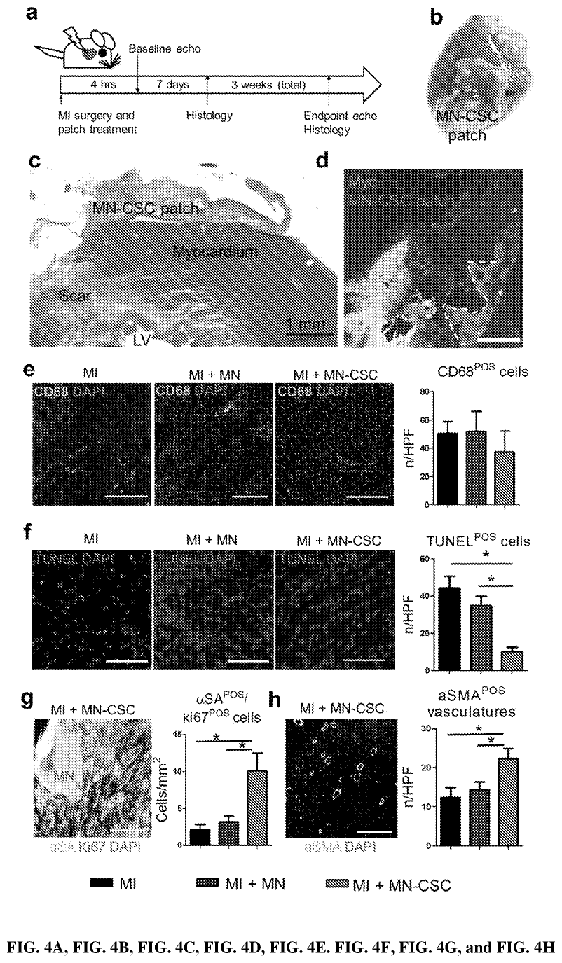

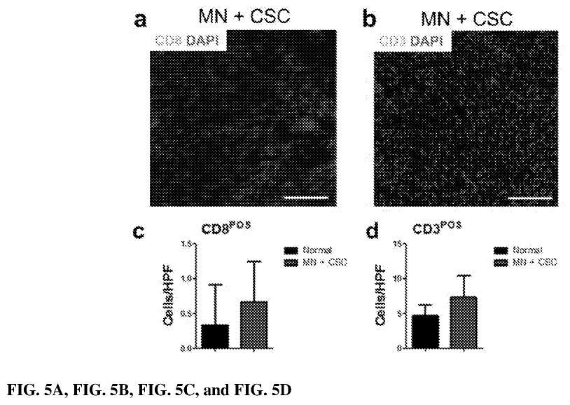

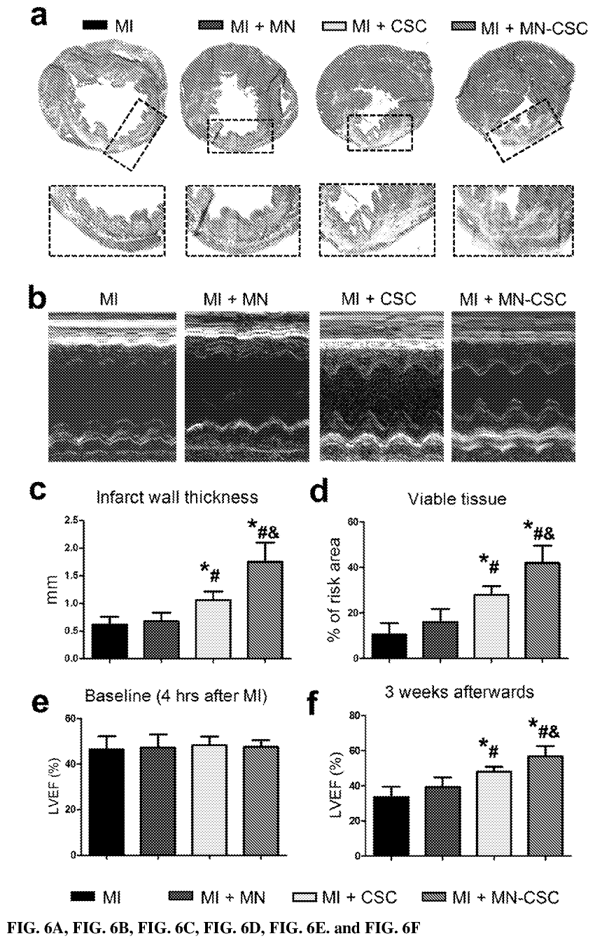

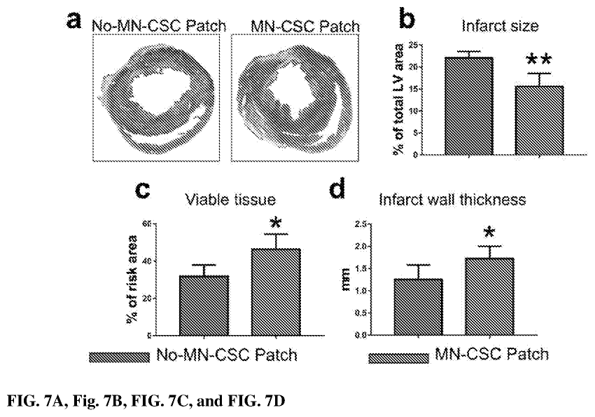

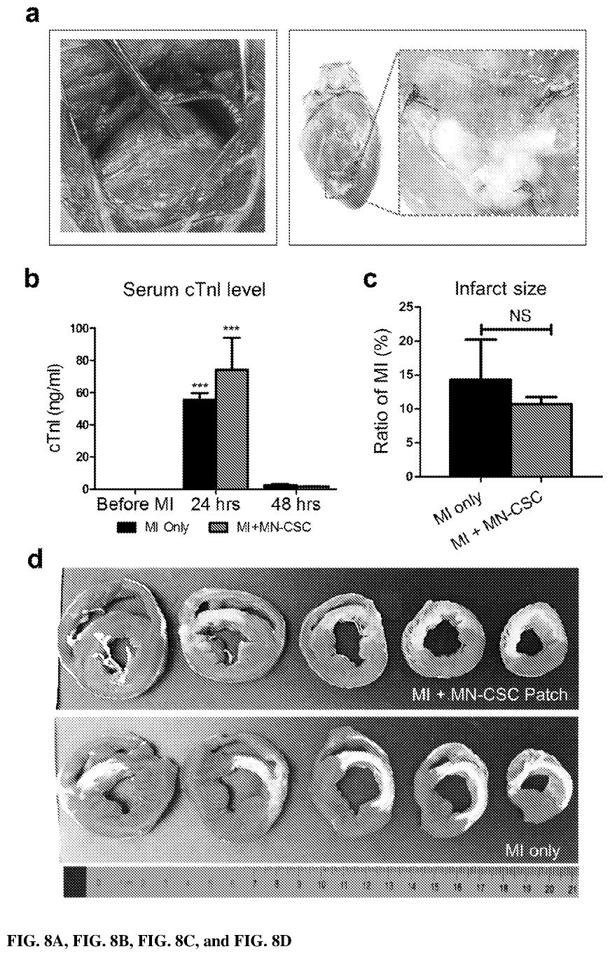

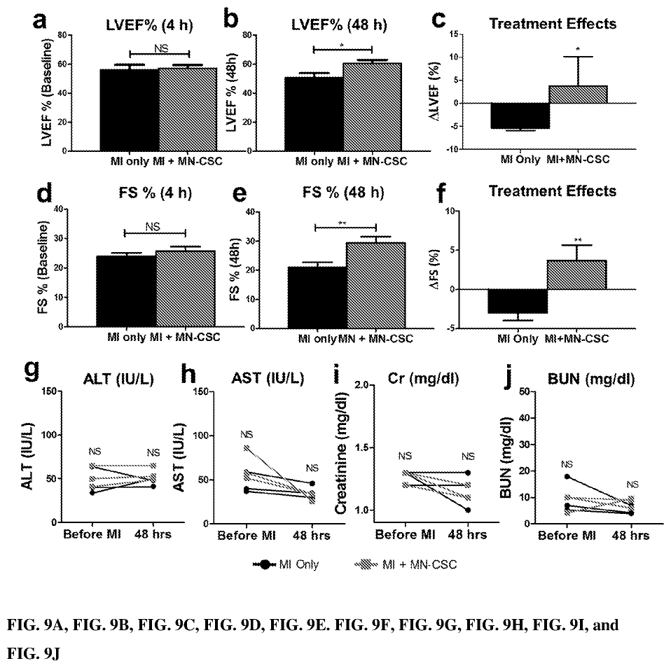

Next, the MN-CSC patch was tested in rats with acute MI (FIG. 4a) Immediately after left anterior descending (LAD) artery ligation, a 0.5×0.5 cm² MN-CSC patch containing 1×10⁶ rat CSCs was laid on the MI area (FIG. 4b). Hematoxylin and eosin (H&E) staining results indicated the MN-CSC patch was on the surface of infarcted heart on day 7 (FIG. 4c). Cy5.5-labeled MNs can be readily detected on the heart 7 days after the transplantation (FIG. 4d). Furthermore, the tissue densities of CD68-positive macrophages were identical among all three groups (FIG. 4e), indicating the MN-CSC patch did not exacerbate inflammation in the post-MI heart. Additionally, no evident CD3- or CD8-positive T infiltration was observed in the hearts treated with MN-CSC on day 7 (FIG. 5). Indeed, treatment with the MN-CSC patch reduced myocardial apoptosis (FIG. 4f), and promoted myocyte proliferation (FIG. 4g) and angiogenesis (FIG. 4h). Masson's trichrome staining revealed morphology and fibrosis of heart 3 weeks after various treatments (FIG. 6a, red: viable tissue, blue: scar). M-mode echocardiographic images showed the LV wall motion after different treatments (FIG. 6b). MN-CSC transplantation increased infarct wall thickness (FIG. 6c) and viable tissue in the risk area (FIG. 6d). As a cardiac function indicator, left ventricular ejection fractions (LVEFs) were determined by echocardiography at baseline (4 hrs after MI) and three weeks afterwards. The LVEFs at baseline were indistinguishable among all three groups, indicating a uniform degree of initial injury (FIG. 6e). 3 weeks after treatment, the hearts received MN-CSC transplantation had the greatest LVEFs (FIG. 6f). The empty MN patch generated neither beneficial nor detrimental effects in the post-MI heart as compared to the MI control group. Additional rat studies were performed to compare the treatment effects from a PVA patch with CSCs but no microneedles (No-MN-CSC group) and a microneedle CSC patch (MN-CSC). Masson's trichrome staining revealed heart morphology and fibrosis 3 weeks after treatment (FIG. 7a; red=viable tissue, blue=scar tissue). Compared to the non-microneedle control, infarcted sizes were effectively controlled by MN-CSC patch transplantation (FIG. 7b). Also, viable tissue in the risk area (FIG. 7c) and infarct wall thickness (FIG. 7d) were increased by MN-CSC patch treatment. The LVEFs at baseline 4 hours were indistinguishable between these two groups, indicating a similar degree of initial injury. 3 weeks after treatment, the hearts received MN-CSC transplantation had a higher LVEFs. To address the toxicity of PVA patches, serum ALT and AST (for liver functions) and creatinine and BUN levels (for kidney functions) 21 days after patch transplantation were measured. The result indicated that patch treatment did not induce any hepatic or renal function impairment. Taken together, these results indicate that the microneedles on CSC patches are indispensable for the full therapeutic benefit. The microneedles provide a pathway for molecules to diffuse from the CSCs to the site of injury (FIGS. 3c and 4d) more efficiently than the non-microneedle controls.

(4) MN-CSC Therapy in a Porcine Model of Acute MI

Despite the widespread use of rodent models for cellular cardiomyopathy, the use of the porcine model is well-justified as the pig heart has human-like myocardial blood flow, ventricular mechanics, and dimensions. Therefore, a pilot porcine study was conducted on MN-CSC cardiac patch. The safety and preliminary therapeutic efficacy were determined in pigs with acute MI induced by LAD ligation (FIG. 8a). Successful induction of MI was verified by the elevation of ST segment on ECG and cardiac troponin I (cTnl) level in serum (FIG. 8b). TTC staining of pig heart sections indicated the infarct area 48 hrs post MI (FIG. 8d). The infarct sizes were indistinguishable between the two groups (FIG. 8c). The baseline (4 h post-MI)—and endpoint (48 h post-infarct)—LVEFs were measured as indicators of cardiac functions in both groups. LVEFs were similar at baseline for both control and MN-CSC patch group, which indicated a similar degree of initial injury (FIG. 9a). However, MN-CSC treated group exhibited greater LVEFs than those from the control group at 48 hrs (FIG. 9b). Left ventricular fractional shortening (LVFS) showed the same trend (FIGS. 9d and 9e). Treatment effects, i.e. the changes in LVEF and LEFS from the baseline to the endpoint, were also calculated. While Control group displayed a functional decline, treatment with MN-CSC patches preserved cardiac functions (FIGS. 9c and 9f). To address the toxicity concerns of MN-CSC patch, blood was collected and tested at baseline (before MI surgery) and 48 hours after MI. The expressions of ALT and AST indicated that liver functions were not impaired by MN-CSC patch transplantation (FIGS. 9g and 9h). Also, the Creatinine and BUN analysis indicated that the MN-CSC patch treatment did not induce any renal function impairment (FIGS. 9i and 9j). Taken together, it was concluded that MN-CSC patch transplantation after acute MI can contribute to the preservation of cardiac function without inducing toxicity.

b) Discussion

“A bandage for a broken heart” has always been a dream for cardiologists and tissue engineers. Here therapeutic cardiac stem cells were integrated with a MN array to form an innovative “cardiac patch”. This is the first study to employ the MN strategy to aid the trans-epicardial delivery of stem cell therapeutics in situ secreted to the heart. Unlike conventional cardiac patches, the prickly MNs can serve as the communication channels between the transplanted stem cells and the host myocardium. Poly (vinyl alcohol) has been widely used to fabricated hydrogels for medical application because of its excellent biocompatibility. In addition, PVA is utilized for MN fabrication here because of its high mechanical strength in dry state and its ability to transport solute in gel state after inserted to skins, where PVA MN can maintain its integrity as a gel within a short term and eventually get dissolved and absorbed by skins during a long period. Thus, the slow dissolving rate of PVA also provides sustained release of regenerative factors from the embedded CSCs. While degradable polymers are advantageous for biomedical applications, the degradation by-products and fragmentation of such polymers can cause side effects. Thus, in one aspect, the patch can comprise degradable or non-degradable polymers.

The in vitro experiment emulated the placement of the MN-CSC patch on the surface of a heart, to answer two questions: 1) Can the media support CSC growth in the integrated device; 2) Can CSC-derived factors be released into the media underneath (FIG. 3). The viability of CSCs cultured on the MN patch was not compromised, indicating neither the PVA nor the fibrin gel was toxic to the cells. Further, CSC-secreted factors were able to diffuse through the needles into the media. Cardiomyocytes (NRCMs) were then added in the media underneath the MN-CSC patch. The viability of cardiomyocytes was not affected by the placement of the MN-CSC patch. Instead, co-culturing with the MN-CSC patch promoted cardiomyocyte proliferation and beating. This was consistent with the observation when cardiomyocytes were co-cultured with CSCs.

The in vivo biocompatibility of the MN-CSC patch was evaluated in rats with acute MI. Syngeneic rat CSCs were used to avoid rejection of the transplanted cells. Nevertheless, foreign body reactions can be triggered against the materials and structures of the MN patch. It was confirmed that the MN-CSC can be successfully placed onto the surface of the heart without the deconstruction of the needles (FIG. 4). Moreover, the insertion of microneedles (400-500 um) caused negligible cardiac tissue damage (FIG. 4) as the fibrin glue applied on the microneedle-side not only created adhesion but also repaired the minor wound during microneedle penetration. The myocardial tissue density of CD68-positive macrophages was indistinguishable among the groups (FIG. 4). And there were no evident infiltrations of CD3/CD8-positive T cells in the heart treated with MN-CSC (FIG. 5). Additionally, the normal expression of ALT, AST, BUN and Creatinine in the pig model treated with MN-patch, confirmed the safety and biocompatibility of the cardiac patch (FIG. 9). This was consistent with the notion that PVA is a material with great biocompatibility.

Next, the hypothesis that transplanted MN-CSC patch can serve as a mini-drug plant for sustained release of regenerative factors was tested. Previous reports indicated that transplantation of CSCs stimulated endogenous repair, by reducing apoptosis and promoting angiomyogenesis. However, such effects were not long-lasting due to the poor retention/engraftment of CSCs. Such caveats were overcome by the present MN-CSC strategy. The transplantation of a MN-CSC patch to rat MI models robustly reduced myocardial apoptosis but increased the numbers of cycling cardiomyocytes and vasculatures in the peri-infarct area. Furthermore, such protective and regenerative effects at the cellular level translated into the improvement of overall heart morphology and pump function (FIG. 6). The hearts received the MN-CSC patch had the greatest wall thicknesses, viable tissues, and LVEFs. Following an acute MI, the heart could undergo severe remodeling which includes the thinning of LV wall, the replacement of health myocardium with scar, and the continuous deterioration of cardiac function. Transplantation of a MN-CSC patch was able to alter this trajectory of maladaptive remolding but promote cardiac regeneration. Moreover, the preliminary safety and efficacy of MN-CSC cardiac patch were evaluated in a pig model of acute MI. The result supported the notion that MN-CSC patch transplantation can preserve cardiac pump function (FIG. 9).

It is worth noting that the MN-CSC patch was delivered through open-chest surgery. In the future, minimal-invasive approaches can be exploded to deploy such patch on the surface of the heart. Further studies can focus on the design of “smart” MN patches that release stem factors in response to physiological environmental stimulus in the post MI heart.

c) Methods

(1) Derivation of Rat CSCs

CSCs were derived from the hearts of WKY rats. Myocardial specimens harvested from WKY rats were cut into fragments of 2 mm³, washed with phosphate-buffered saline, and partially digested with collagenase (Sigma-Aldrich). The tissue fragments were cultured as cardiac explants on a 0.5 mg/ml fibronectin (Corning, Corning, N.Y., USA) solution coated surface in Iscove's modified Dulbecco's medium (Invitrogen, Carlsbad, Calif., USA) containing 20% fetal bovine serum (Corning). A layer of stromal-like cells emerged from the cardiac explant with phase-bright cells over them. The explant-derived cells were harvested using TryPEL Select (Gibco). Harvested cells were seeded at a density of 2×10⁴ cells/ml in Ultra Low Attachment flasks (Corning, Corning, N.Y.) for cardiosphere formation. In about one week, explant-derived cells spontaneously aggregated into cardiospheres. The cardiospheres were collected and plated onto fibronectin-coated surfaces to generate cardiosphere-derived CSCs. All cultures were incubated in 5% CO₂ at 37° C.

(2) Fabrication and Characterization of MNs

All of the MNs in this study were fabricated using five uniform silicone molds from Blueacre Technology Ltd. Each MN had a round base of 300 μm in diameter, which tapers over a height of 600 μm to a tip radius of around 5 μm. The MNs were arranged in a 20×20 array with 600 μm tip-tip spacing. First, PVA aqueous solution (10 wt %, 100 μL) was prepared and deposited in a silicone mold, which was kept under reduced vacuum for 20 minutes and then transferred to a Hettich Universal 32 R centrifuge for 20 min at 500 μm to compact gel solution into MN cavities. Then, additional aqueous solutions of PVA (100 μL) was loaded into mold and the above procedure was repeated for several times until 500 μL PVA solution in total was added to mold. Finally, each micromold was dried under vacuum for another 24 hours. After the desiccation, the MN array patch was carefully separated from the silicone mold for further application. The morphology of the MNs was characterized using a FEI Verios 460L field-emission scanning electron microscope. Cy5.5 labeled PVA MN was prepared similarly. In short, Cy5.5 was first used to modify PEG_{5K}-NH₂, which was subsequently dissolved in PVA aqueous solution to label the MN. The modification of Cy5.5 with PEG_{5K} can increase its water solubility and increase its retention in MN. The mechanical strength of MNs with a stress-strain gauge was determined by pressing a stainless steel plate toward MNs on a DTS delaminator. The initial gauge between the tips of MN and the plate was 2.00 mm, with the load cell capacity of 10.00 N. The plate approaching MNs at a speed of 0.1 mm/s. The force led to the failure of MNs was defined as the force at which the needle began to buckle.

(3) Preparation and Culture of MN-CSC

CSCs were encapsulated in fibrin gel (Baxter Healthcare Corp) and placed on the basal surface of MN, and thus formed MN-CSC. In vitro, MN-CSC was cultured on 4-well chamber with 20% FBS media. Cell viability was evaluated by Live/Dead Viability/Cytotoxicity Kit on day 1, day 3 and day 7. For confocal image, DiO-labeled CSCs was encapsulated in fibrin gel and placed on the basal surface of Cy5.5 MN. After 3-day culture, fibrin gel encapsulating CSCs were taken off and the left MN was imaged under confocal fluorescent microscope (ZEISS LSM 880).

(4) In Vitro Cytokine Release

1×10⁵rat CSCs or MN-CSC containing 1×10⁵rat CSCs were cultured in 1 ml of FBS-free media in a 24-well plate. Conditioned media was collected from the plates on days 1, 3, and 7, to study the cells' release of growth factors. The concentrations of various growth factors in the media were measured by ELISA kits (R&D Systems, Minneapolis, MN; B-Bridge International, Cupertino, Calif.), per the manufacturer's instructions. The growth factors assayed were hepatocyte growth factor (HGF), vascular endothelial growth factor (VEGF), and insulin-like growth factor (IGF-1).

(5) Culture of Neonatal Rat Cardiomyocytes with MN-CSC

Neonatal rat cardiomyocytes (NRCMs) were derived from SD rats. NRCMs were cultured in a 4-well chamber. A MN or MN-CSC patch was placed on the surface of NRCMs. A solitary NRCM culture was included as a control. A Live/Dead Viability/Cytotoxicity Kit was used to determine the cell viability of NRCMs at day 3. The morphology of the cells was characterized using NIH Image J software. Cell proliferation was evaluated by the percentage of α-SA/ki67 positive cardiomyocytes by immunocytochemistry staining.

(6) Immunogenicity Studies on MN-CSC

SD rats were anaesthetized with Ketamine. Then, their hearts were exposed by left thoracotomy under sterile conditions. An MN-CSCs patch, containing 1×10⁶ CSCs, was placed onto the heart. Those rats that underwent the left thoracotomy but did not receive a patch were set as normal control. After 7 days, all rats were sacrificed, and hearts were collected and cryopreserved in optimum cutting temperature (OCT) compound. They were then cryo-sectioned and fixed with 4% paraformaldehyde. Protein Block Solution (DAKO, Carpinteria, Calif.) containing 0.1% saponin (Sigma, St Louis, Mo.), was used to permeabilize and protein-block each section. The following primary antibodies were used to target the desired proteins after an overnight incubation at 4° C.: mouse anti-CD8 alpha (1:100, mca48r, abd Serotec, Raleigh, N.C.) and rabbit anti-CD3 (1:100, ab16669, Abcam). Subsequently, FITC secondary antibodies (1:100; Abcam) were used for the detection of primary antibodies. DAPI (Life Technology, NY, USA) was used to counter-stain cell nuclei. Images were taken with an Olympus epi-fluorescence microscope.

(7) Rat Model of Myocardial Infarction

All animal work was compliant with the Institutional Animal Care and Use Committee at North Carolina State University. Briefly, SD rats were anaesthetized with Ketamine Under sterile conditions, the heart was exposed by left thoracotomy and acute MI was produced by permanent ligation of the left anterior descending (LAD) coronary artery. Immediately after MI induction, the heart was randomized to receive one of the following four treatment arms: (1) MI group: MI induction without any treatment; (2) MI +MN group: MN patch was placed onto the surface of infarcted heart; (3) MI +CSC group: 1x10⁶ CSCs encapsulated in fibrin gel was placed onto the infarcted heart; (4) MI +MN-CSC group: MN-CSCs patch containing 1×10⁶CSCs was placed onto the infarcted heart. Before patch application, fibrin glue was applied on the microneedle-side of the patch to aid adhesion. Then, the patch was placed on the heart with a gentle pressure by tweezer tips for 30 sec. Heart contraction generated counter-acting force against tweezer tips and led to the insertion of microneedles (FIG. 1b) into the cardiac tissue.

(8) Transplantation of Patches with or Without Microneedles in Rats with MI

SD rats were anaesthetized with Ketamine Immediately after MI induction (48), animals were randomized to receive one of the following 2 treatment: (1) MI+No-MN-CSC group: a PVA patch without microneedles containing 1×10⁶ CSCs was placed onto the infarcted heart; (2) MI+MN-CSC group: a MN-CSC patch containing 1×10⁶ CSCs was placed onto the infarcted heart. Serum ALT, AST, Creatinine and BUN levels were measured 21 days after transplantation. Normal range for rats: ALT=10˜40 IU/L, AST=50˜150 IU/L (49), Creatinine=0.1˜0.55 mg/dL, BUN=3.56˜25.43 mg/dL (50).

(9) Pig Model of Myocardial Infarction

All animal work is compliant with Institutional Animal Care and Usage Committee at North Carolina State University. Acute MI was induced in female Yorkshire-pigs (50-70 lbs) by permanent ligation of LAD during an open-chest surgery. 20 min after ischemia, a 2.5 cm×2.5 cm MN-CSC cardiac patch was sutured on the heart surface to cover the MI injured area that downstream of LAD (Silkam 2/0, B/Braun Suture). The control animals received no cardiac patches. After the procedures, all animals were closely monitored by NCSU veterinary services staff and then the pigs were euthanized 48 h post MI injury. LVEFs were determined by echocardiography using a Philips CX30 ultrasound system coupled with an S4-2 high-frequency probe at two time points (4 h and 48 h post-MI). Blood was collected before MI, 24 h and 48 h post MI. Infarct area of LV myocardium was traced through the digital images of TTC staining (5 slices) and measured by ImageJ analysis. Then, the infarct ratio was measured and calculated as:

Infarct Size=Σₙ₌₁⁵(Slice Infarcted area %×Slice weight)/Heart Weight×100%.

(10) Triphenyl Tetrazolium Chloride (TTC) Assay

TTC assay was performed to differentiate the active cardiac tissue and the inactive infarct cardiac tissue. A sterilized solution of 2,3,5-Triphenyl Tetrazolium Chloride (TTC) was made by dissolving TTC (2 g; MP Biomedicals, LLC) into 200 ml of sterilized PBS and then pre-warmed at 37° C. incubator for 30mins. The heart was collected and washed with sterilized PBS and then placed in freezer until the heart became stiff. Five 7 mm sections were cut from Apex to bottom and incubate in pre-warmed TTC solution at 37° C. for 30 mins. Afterwards, the sections were fixed in 10% formaldehyde solution for 2 hours.

(11) Cardiac Function Assessment

All animals underwent transthoracic echocardiography under 2.0% isofluorane-oxygen mixture anesthesia in supine position at 4 h and 3 weeks. The procedure was performed by an animal cardiologist blind to the experimental design using a Philips CX30 ultrasound system coupled with an L15 high-frequency probe. Hearts were imaged in 2D in long-axis views at the level of the greatest LV diameter. EF was determined by using the formula (LVEDV−LVESV/LVEDV)×100%. Two-dimensional guided M-mode images at chordae tendineae level were captured.

(12) Heart Morphometry

All animals were sacrificed 3 weeks post MI induction, after the echocardiography study. Hearts were collected and cryopreserved in optimum cutting temperature (OCT) compound. 10 μm-thick heart sections were cut from the apex to the height of the ligation. Each section was cut at 100 μm intervals. Masson's trichrome (HT15 Trichrome Staining (Masson) Kit; Sigma-Aldrich) staining was performed according to the manufacturer's specifications. A PathScan Enabler IV slide scanner (Advanced Imaging Concepts, Princeton, N.J.) was used to acquire images of each stained section. These were used to assess morphometric parameters (i.e. infarct thickness and viable myocardium) which were quantified with NIH ImageJ software.

(13) Histology

For immunohistochemistry staining, heart cryosections were fixed with 4% paraformaldehyde, permeabilized and blocked with Protein Block Solution (DAKO, Carpinteria, Calif.) containing 0.1% saponin (Sigma, St Louis, Mo., and then incubated with the following antibodies overnight at 4° C.: mouse anti-alpha sarcomeric actin (1:100, a7811, Sigma), mouse anti-CD68 (1:100, ab955, Abcam), mouse anti-Actin, a-Smooth Muscle antibody (1:100, A5228, Sigma), and rabbit anti-Ki67 (1:100, ab15580, Abcam). FITC- or Texas-Red secondary antibodies (1:100) were obtained from Abcam Company and used for the conjunction with these primary antibodies. For assessment of cell apoptosis, heart cryosections were incubated with TUNEL solution (Roche Diagnostics GmbH, Mannheim, Germany) and counterstained with DAPI (Life Technology, NY, USA). Images were taken by an Olympus epi-fluorescence microscopy system.

For Haematoxylin and Eosin (H&E) staining, sections were fixed in Hematoxylin (Sigma-Aldrich, MO, USA) for 5 min at room temperature, and then rinsed for 2 minutes in running water. The sections were then dipped in acid alcohol for 2 seconds, in sodium bicarbonate (5 dips), and in dehydrant (Richard-Allan Scientific, MI, USA) for 30 seconds. They were subsequently submerged in Eosin (Sigma-Aldrich, MO, USA) for 2 minutes and thoroughly washed in dehydrant and Xylene (VWR, PA, USA).

(14) Statistical Analysis

All results are expressed as mean ±s.d. Comparison between two groups was performed with two-tailed Student's t-test. Comparisons among more than two groups were performed using one-way ANOVA followed by post hoc Bonferroni test. Differences were considered statistically significant when the P value <0.05.

### D. REFERENCES

A. J. Boyle, S. P. Schulman, J. M. Hare, Stem cell therapy for cardiac repair: Ready for the next step, Circulation 114, 339-352 (2006).
A. S. Hickey, N. A. Peppas, Mesh size and diffusive characteristics of semicrystalline poly(vinyl alcohol) membranes prepared by freezing/thawing techniques, J. Memb. Sci. 107, 229-237 (1995).
A. Vandergriff, K. Huang, D. Shen, S. Hu, M. T. Hensley, T. G. Caranasos, L. Qian, K. Cheng, Targeting regenerative exosomes to myocardial infarction using cardiac homing peptide, Theranostics 8, 1869-1878 (2018).
C. Chiappini, E. De Rosa, J. O. Martinez, X. Liu, J. Steele, M. M. Stevens, E. Tasciotti, Biodegradable silicon nanoneedles delivering nucleic acids intracellularly induce localized in vivo neovascularization, Nat. Mater. 14, 532-539 (2015).
C. E. Maclas, H. Bodugoz-Senturk, O. K. Muratoglu, Quantification of PVA hydrogel dissolution in water and bovine serum, Polym. (United Kingdom) 54, 724-729 (2013).
C. M. Hassan, J. H. Ward, N. A. Peppas, Modeling of crystal dissolution of poly(vinyl alcohol) gels produced by freezing/thawing processes, Polymer (Guildf). 41,6729-6739 (2000).
C. P. Hodgkinson, A. Bareja, J. A. Gomez, V. J. Dzau, Emerging Concepts in Paracrine Mechanisms in Regenerative Cardiovascular Medicine and Biology, Circ. Res. 118,95-107 (2016).
D. Shen, J. Tang, M. T. Hensley, T. Li, T. G. Caranasos, T. Zhang, Effects of Matrix Metalloproteinases on the Performance of Platelet Fibrin Gel Spiked With Cardiac Stem Cells in Heart Repair, Stem Cells Transl. Med. 5,793-803 (2016).
E. J. Benjamin, M. J. Blaha, S. E. Chiuve, M. Cushman, S. R. Das, R. Deo, S. D. De Ferranti, J. Floyd, M. Fornage, C. Gillespie, C. R. Isasi, M. C. Jim'nez, L. C. Jordan, S. E. Judd, D. Lackland, J. H. Lichtman, L. Lisabeth, S. Liu, C. T. Longenecker, R. H. MacKey, K. Matsushita, D. Mozaffarian, M. E. Mussolino, K. Nasir, R. W. Neumar, L. Palaniappan, D. K. Pandey, R. R. Thiagarajan, M. J. Reeves, M. Ritchey, C. J. Rodriguez, G. A. Roth, W. D. Rosamond, C. Sasson, A. Towfghi, C. W. Tsao, M. B. Turner, S. S. Virani, J. H. Voeks, J. Z. Willey, J. T. Wilkins, J. H. Y. Wu, H. M. Alger, S. S. Wong, P. Muntner, Heart Disease and Stroke Statistics'2017 Update: A Report from the American Heart Association (2017).
E. Marban, Breakthroughs in Cell Therapy for Heart Disease: Focus on Cardiosphere-Derived Cells, Mayo Clin Proc. 89,850-858 (2014).
G. Lanzoni, T. Oikawa, Y. Wang, C. Cui, G. Carpino, V. Cardinale, D. Gerber, M. Gabriel, J. Dominguez-Bendala, M. E. Furth, E. Gaudio, D. Alvaro, L. Inverardi, L. M. Reid, Clinical programs of stem cell therapies for liver and pancreas, Stem Cells 31,1-28 (2013).
G. Paradossi, F. Cavalieri, E. Chiessi, C. Spagnoli, Poly(vinyl alcohol) as versatile biomaterial for potential biomedical applications., J Mater Sci Mater Med. 14,687-91 (2003).
I. J. Fox, G. Q. Daley, S. A. Goldman, J. Huard, T. J. Kamp, Use of differentiated pluripotent stem cells in replacement therapy for treating disease, Science (80). 345,889-901 (2014).
J. Tang, A. Vandergriff, Z. Wang, M. T. Hensley, J. Cores, T. A. Allen, P.-U. Dinh, J. Zhang, T. G. Caranasos, K. Cheng, A Regenerative Cardiac Patch Formed by Spray Painting of Biomaterials onto the Heart, Tissue Eng. Part C Methods 23,146-155 (2017).

J. Tang, D. Shen, T. G. Caranasos, Z. Wang, A. C. Vandergriff, T. A. Allen, M. T. Hensley, P. U. Dinh, J. Cores, T. S. Li, J. Zhang, Q. Kan, K. Cheng, Therapeutic microparticles functionalized with biomimetic cardiac stem cell membranes and secretome, Nat. Commun. 8, 1-9 (2017).
J. Tang, T. Su, K. Huang, P. U. Dinh, Z. Wang, A. Vandergriff, M. T. Hensley, J. Cores, T. Allen, T. Li, E. Sproul, E. Mihalko, L. J. Lobo, L. Ruterbories, A. Lynch, A. Brown, T. G. Caranasos, D. Shen, G. A. Stouffer, Z. Gu, J. Zhang, K. Cheng, Targeted repair of heart injury by stem cells fused with platelet nanovesicles, Nat. Biomed. Eng. 2,17-26 (2018).
J. Tang, X. Cui, T. G. Caranasos, M. T. Hensley, A. C. Vandergriff, Y. Hartanto, D. Shen, H. Zhang, J. Zhang, K. Cheng, Heart Repair Using Nanogel-Encapsulated Human Cardiac Stem Cells in Mice and Pigs with Myocardial Infarction, ACS Nano 11,9738-9749 (2017).
J. Walter, L. B. Ware, M. A. Matthay, Mesenchymal stem cells: Mechanisms of potential therapeutic benefit in ARDS and sepsis, Lancet Respir. Med. 2,1016-1026 (2014).
J. Yu, Y. Zhang, W. Sun, A. R. Kahkoska, J. Wang, J. B. Buse, Z. Gu, Insulin-Responsive Glucagon Delivery for Prevention of Hypoglycemia, Small 13,1-5 (2017).
J. Yu, Y. Zhang, Y. Ye, R. DiSanto, W. Sun, D. Ranson, F. S. Ligler, J. B. Buse, Z. Gu, Microneedle-array patches loaded with hypoxia-sensitive vesicles provide fast glucose-responsive insulin delivery., Proc. Natl. Acad. Sci. 112,8260-8265 (2015).
J. Yu, Y. Zhang, Z. Gu, Glucose-Responsive Insulin Delivery by Microneedle-Array Patches Loaded with Hypoxia-Sensitive Vesicles. Methods Mol Biol. (2017), vol. 1570, pp. 251-259.
K. Cheng, A. Ibrahim, M. T. Hensley, D. Shen, B. Sun, R. Middleton, W. Liu, R. R. Smith, E. Marban, Relative Roles of CD90 and c-Kit to the Regenerative Efficacy of Cardiosphere-Derived Cells in Humans and in a Mouse Model of Myocardial Infarction, J. Am. Heart Assoc. 3, e001260-e001260 (2014).
K. Cheng, D. Shen, M. T. Hensley, R. Middleton, B. Sun, W. Liu, G. De Couto, E. Marban, Magnetic antibody-linked nanomatchmakers for therapeutic cell targeting, Nat. Commun. 5 (2015), doi:10.1038/ncomms5880.
K. Cheng, K. Malliaras, D. Shen, E. Tseliou, V. Ionta, J. Smith, G. Galang, B. Sun, C. Houde, E. Marban, Intramyocardial Injection of Platelet Gel Promotes Endogenous Repair and Augments Cardiac Function in Rats With Myocardial Infarction, J. Am. Coll. Cardiol. 59,256-264 (2012).
K. Cheng, K. Malliaras, R. R. Smith, D. Shen, B. Sun, A. Blusztajn, Y. Xie, A. Ibrahim, M. A. Aminzadeh, W. Liu, T.S. Li, M. A. De Robertis, L. Marban, L. S. C. Czer, A. Tre, Human Cardiosphere-Derived Cells From Advanced Heart Failure Patients Exhibit Augmented Functional Potency in Myocardial Repair, JACC Hear. Fail. 2, 49-61 (2014).
K. M. M. Hasan, N. Tamanna, M. A. Hague, Biochemical and histopathological profiling of Wistar rat treated with Brassica napus as a supplementary feed, Food Sci. Hum. Wellness 7, 77-82 (2018).
K. U. Hong, Q. H. Li, Y. Guo, N. S. Patton, A. Moktar, A. Bhatnagar, R. Bolli, A highly sensitive and accurate method to quantify absolute numbers of c-kit+cardiac stem cells following transplantation in mice, Basic Res. Cardiol. 108, 346 (2013).
K. U. Hong, R. Bolli, Cardiac stem cell therapy for cardiac repair, Curr. Treat. Options Cardiovasc. Med. 16 (2014), doi:10.1007/s11936-014-0324-3.
L. E. Lillie, N. J. Temple, L. Z. Florence, Reference values for young normal Sprague-Dawley rats: weight gain, hematology and clinical chemistry, Hum. Exp. Toxicol. 15, 612-616 (1996).
L. Luo, J. Tang, K. Nishi, C. Yan, P. U. Dinh, J. Cores, T. Kudo, J. Zhang, T. S. Li, K. Cheng, Fabrication of Synthetic Mesenchymal Stem Cells for the Treatment of Acute Myocardial Infarction in Mice, Circ. Res. 120, 1768-1775 (2017).
M. C. Chen, Z. W. Lin, M. H. Ling, Near-infrared light-activatable microneedle system for treating superficial tumors by combination of chemotherapy and photothermal therapy, ACS Nano 10, 93-101 (2016).
M. Li, J. C. Izpisua Belmonte, Mending a Faltering Heart, Circ. Res. 118, 344-351 (2016).
M. R. Prausnitz, Microneedles for transdermal drug delivery, Adv. Drug Deliv. Rev. 56, 581-587 (2004).
N. A. Peppas, A. Khademhosseini, Make better, safer biomaterials, Nature. 540, 7633 (2016).
N. A. Peppas, A. Khademhosseini, Make better, safer biomaterials, Nature. 540, 7633 (2016).
O. Lindvall, Z. Kokaia, Stem cells for the treatment of neurological disorders, Nature 441, 1094-1096 (2006).
O. Olatunji, D. B. Das, M. J. Garland, L. Belaid, R. F. Donnelly, Influence of array interspacing on the force required for successful microneedle skin penetration: Theoretical and practical approaches, J. Pharm. Sci. 102,1209-1221 (2013).
P. A. R. Bolli, A. R. Chugh, D. D' Amario, J. H. Loughran, M. F. Stoddard, S. Ikram, G. M. Beache, S. G. Wagner, A. Leri, T. Hosoda, J. B. Elmore, P. Goihberg, D. Cappetta, N. K. Solankhi, I. Fahsah, D. G. Rokosh, M. S. Slaughter, J. Kajstura, Effect of cardiac stem cells in patients with ischemic cardiomyopathy: initial results of the SCIPIO Trial., Can. J. Cardiol. 378, 1847-1857 (2011).
P. S. Jhund, J. J. V. McMurray, Heart failure after acute myocardial infarction a lost battle in the war on heart failure?, Circulation 118, 2019-2021 (2008).
R. Lakshmanan, P. Kumaraswamy, U. M. Krishnan, S. Sethuraman, Engineering a growth factor embedded nanofiber matrix niche to promote vascularization for functional cardiac regeneration, Biomaterials 97, 176-195 (2016).
R. Madonna, L. W. Van Laake, S. M. Davidson, F. B. Engel, D. J. Hausenloy, S. Lecour, J. Leor, C. Perrino, R. Schulz, K. Ytrehus, U. Landmesser, C. L. Mummery, S. Janssens, J. Willerson, T. Eschenhagen, P. Ferdinandy, J. P. G. Sluijter, Position Paper of the European Society of Cardiology Working Group Cellular Biology of the Heart: Cell-based therapies for myocardial repair and regeneration in ischemic heart disease and heart failure, Eur. Heart J. 37, 1789-1798 (2016).
R. Madonna, P. Ferdinandy, R. De Caterina, J. T. Willerson, A. J. Marian, Recent developments in cardiovascular stem cells, Circ. Res. 115, e71-e78 (2014).
R. R. Makkar, R. R. Smith, K. Cheng, K. Malliaras, L. E. J. Thomson, D. Berman, L. S. C. Czer, L. Marban, A. Mendizabal, P. V. Johnston, S. D. Russell, K. H. Schuleri, A. C. Lardo, G. Gerstenblith, E. Marban, Intracoronary cardiosphere-derived cells for heart regeneration after myocardial infarction (CADUCEUS): A prospective, randomised phase 1 trial, Lancet 379,895-904 (2012).
S. B. Seif-Naraghi, J. M. Singelyn, M. A. Salvatore, K. G. Osborn, J. J. Wang, U. Sampat, O. L. Kwan, G. M. Strachan, J. Wong, P. J. Schup-Magoffin, R. L. Braden, K. Bartels, J. A. DeQuach, M. Preul, A. M. Kinsey, A. N. DeMaria, N. Dib, K. L. Christman, Safety and Efficacy of an Injectable Extracellular Matrix Hydrogel for Treating Myocardial Infarction, Sci. Transl. Med. 5, 173ra25 LP-173ra25 (2013).
S. Li, X. H. Yang, Fabrication and characterization of electrospun wool keratin/poly(vinyl alcohol) blend nanofibers, Adv. Mater. Sci. Eng. 2014 (2014), doi:10.1155/2014/163678.
S. P. Davis, B. J. Landis, Z. H. Adams, M. G. Allen, M. R. Prausnitz, Insertion of microneedles into skin: Measurement and prediction of insertion force and needle fracture force, J. Biomech. 37, 1155-1163 (2004).
S. Roura, C. Soler-Botija, J. R. Bag'O, A. A.-Valldeperas, M. A. F'ernandez, C. G'alvez-Mont'on, C. Prat-Vidal, I. Perea-Gil, J. Blanco, Postinfarction Functional Recovery Driven by a 3D Engineered Fibrin Patch Composed of Human Umbilical Cord Blood-Derived Mesenchymal Stem Cells, Stem Cells Transl. Med. , 956-966 (2015).
T. Noguchi, T. Yamamuro, M. Oka, P. Kumar, Y. Kotoura, S. Hyon, Poly(vinyl alcohol) hydrogel as an artificial articular cartilage: evaluation of biocompatibility, J Appl Biomater. 2, 101-107 (1991).
V. F. M. Segers, R. T. Lee, Stem-cell therapy for cardiac disease, Nature 451, 937-942 (2008).
V. F. Segers, R. T. Lee, Biomaterials to enhance stem cell function in the heart, Circ. Res. 109, 910-922 (2011).
Y. L. Tang, Y. Tang, Y. C. Zhang, K. Qian, L. Shen, M. I. Phillips, Improved graft mesenchymal stem cell survival in ischemic heart with a hypoxia-regulated heme oxygenase-1 vector, J. Am. Coll. Cardiol. 46, 1339-1350 (2005).
Y. Lu, A. A. Aimetti, R. Langer, Z. Gu, Bioresponsive materials, Nat. Rev. Mater. 2, 16075 (2016).
Y. Lu, A. A. Aimetti, R. Langer, Z. Gu, Bioresponsive materials, Nat. Rev. Mater. 2, 16075 (2016).
Y. Ye, C. Wang, X. Zhang, Q. Hu, Y. Zhang, Q. Liu, D. Wen, J. Milligan, A. Bellotti, L. Huang, G. Dotti, Z. Gu, A melanin-mediated cancer immunotherapy patch, Sci. Immunol. 2 (2017)
Y. Ye, J. Wang, Q. Hu, G. M. Hochu, H. Xin, C. Wang, Z. Gu, Synergistic Transcutaneous Immunotherapy Enhances Antitumor Immune Responses through Delivery of Checkpoint Inhibitors, ACS Nano 10, 8956-8963 (2016).
Z. Lin, W. T. Pu, Strategies for Cardiac Regeneration and Repair, Sci. Transl. Med. 6, 239ry 1 LP-239rv1 (2014)

1. A microneedle patch for transport of a material across a biological barrier of a subject comprising:
a) a plurality of microneedles each having a base end and a tip;
b) a substrate to which the base ends of the microneedles are attached or integrated; and
c) a plurality of cardiac precursor cells.

2. The microneedle patch of claim 1, wherein the plurality of microneedles comprises a biocompatible polymer.

3. The microneedle patch of claim 2, wherein the biocompatible polymer comprises polyvinyl alcohol (PVA).

4. The microneedle patch of claim 3, wherein the biocompatible polymer is crosslinked.

5. The microneedle patch of claim 1, wherein the plurality of microneedles have a center-to-center interval of about 200 μm to about 800 μm.

6. The microneedle patch of claim 1, wherein the plurality of microneedles have a height of about 600 nm to 1.8 μm.

7. A method of treating cardiac injury comprising administering to a subject with cardiac injury the microneedle patch of claim 1.

8. A method of locally delivering a cardiac precursor cell to a site of cardiac injury comprising providing a microneedle patch for transport of a material across a biological barrier of a subject and administering the microneedle patch to a subject in need thereof to the site of the cardiac injury; wherein the microneedle patch for transport of the material across a biological barrier comprises:
a) a plurality of microneedles each having a base end and a tip;
b) a substrate to which the base ends of the microneedles are attached or integrated; and
c) a plurality of cardiac precursor cells attached to the basal surface of the microneedle patch.

9. A method of treating a cardiac injury in a subject in need thereof comprising:
a) providing a microneedle patch for transport of a material across a biological barrier of a subject comprising: a plurality of microneedles each having a base end and a tip; a substrate to which the base ends of the microneedles are attached or integrated; and a plurality of cardiac precursor cells attached to the basal surface of the microneedle patch; and
b) administering the microneedle patch to a subject in need of treating cardiac injury.

10. The method of claim 8, wherein the cardiac injury is caused by myocardial infarction, ischemic injury, and ischemic reperfusion injury, pericarditis, acute gastroenteritis, myocarditis, surgery, blunt trauma.

11. The method of claim 8, wherein the administering step b) comprises inserting the microneedle patch onto the surface of the site of cardiac injury.

12. The method of claim 8, wherein the microneedle patch is administered within 48 hours of the injury.
